(19) United States
(12) Patent Application Publication
(10) Pub. No.: US 2022/0387230 A1
(43) Pub. Date: Dec. 8, 2022

(54) Absorbent Article With Cushiony Softness

(71) Applicant: The Procter & Gamble Company, Cincinnati, OH (US)

(72) Inventors: Abhishek Prakash SURUSHE, Kelkheim (DE); Kevin Ronald KANYA, Liberty Township, OH (US); Jeromy Thomas RAYCHECK, South Lebanon, OH (US); Sarah Ann SANBORN, Cincinnati, OH (US); Tanner Laurie WILLIAMS, Loveland, OH (US); Adrien GRENIER, Schwalbach am Taunus (DE)

(21) Appl. No.: 17/333,050

(22) Filed: May 28, 2021

(51) Int. Cl. A61F 13/513; A61F 13/15; A61F 13/551; A61F 13/511

(52) U.S. Cl. CPC A61F 13/513; A61F 13/15203; A61F 13/551; A61F 13/5116; A61F 2013/15406; A61F 2013/51169; A61F 13/42

(57) ABSTRACT

An absorbent article comprising a topsheet, a backsheet, and an absorbent core is provided. A hydroentangled outer cover material is joined to the backsheet and forms a portion of a garment-facing surface of the absorbent article. The outer cover material comprises a bond pattern formed of bond elements. The outer cover material has a basis weight in the range of about 20 gsm to about 35 gsm. A garment-facing surface of the outer cover material has a Kinetic Sliding Force in the range of about 6.0 to about 7.5. The absorbent article comprises a hydroentangled cushion material forming at least a portion of a garment-facing surface of the core wrap or positioned intermediate the core wrap and the backsheet. The cushion material has a basis weight in the range of about 20 gsm to about 40 gsm.

### FIELD

The present disclosure is generally directed to absorbent articles with cushiony softness.

### BACKGROUND

Absorbent articles comprising nonwoven materials are used in the hygiene industry to contain and absorb bodily exudates (i.e., urine, bowel movements, and menses) in infants, toddlers, children, and adults. Absorbent articles may include, but not be limited to, diapers, pants, adult incontinence products, and absorbent pads. Various components of these absorbent articles comprise one or more nonwoven materials. Consumers desire nonwoven materials in absorbent articles to be soft and lofty for the best comfortable wearer experience. Current nonwoven materials for absorbent articles and absorbent articles themselves should be improved to meet this need.

### SUMMARY

The present disclosure provides, in part, nonwoven materials for absorbent articles that provide the absorbent articles with cushiony softness and loft for the best comfortable wearer experience. Parents and caregivers desire a soft and cushiony feel from a garment-facing surface of an absorbent articles. They do not want to feel anything harsh or stiff. As such, the present disclosure provides absorbent articles with certain outer cover materials to at least partially solve this problem. Optionally, the present disclosure also provides absorbent articles with certain cushion layers intermediate the backsheet and an absorbent material of an absorbent core. Such a cushion layer can mask any rough or graining feeling of an absorbent core or of an absorbent material within the absorbent core. As a result, the absorbent articles present a soft and lofty feel from a garment-facing surface thereof.

Absorbent articles are at times packaged under significant compression. As such, it may be difficult to dispense the first few absorbent articles from packages. The outer cover materials of the present disclosure through their composition, texture, stack height of the packages, and bond pattern may enhance absorbent article dispensing, especially from a full and compressed package of absorbent articles. This is true for the absorbent articles of the present disclosure even though the absorbent articles of the present disclosure have outer cover materials having a higher Kinetic Sliding Force as current outer cover materials. As such, Applicants have broken a technical barrier by raising the Kinetic Sliding Force of the outer cover materials while still achieving better dispensing from a compressed package of absorbent articles.

The present disclosure provides, in part, an absorbent article comprising a liquid permeable topsheet, a liquid impermeable backsheet, and an absorbent core positioned at least partially intermediate the topsheet and the backsheet. The absorbent core may be positioned within a core wrap. The absorbent core comprises one or more absorbent materials. A hydroentangled, or otherwise formed, nonwoven outer cover material is joined to the backsheet and forms a portion of a garment-facing surface of the absorbent article. The outer cover material may comprise two or more layers of spunbond. The outer cover material may comprise a bond pattern formed of bond elements, wherein at least some of the bonds may comprise two arcuate portions. The bond pattern may have a bond area of about 8% to about 17%, or about 10% to about 15% relative to an entire area of a surface of the outer cover material. The spunbond of the outer cover material may comprises a softening additive. The outer cover material may have a basis weight in the range of about 20 gsm to about 35 gsm, about 25 gsm to about 35 gsm, about 25 gsm, or about 30 gsm. A garment-facing surface of the outer cover material may have a Kinetic Sliding Force in the range of about 6.0 to about 7.5, according to the Kinetic Sliding Force Test. The absorbent article may comprise a hydroentangled, or otherwise formed, cushion material forming at least a portion of a garment-facing surface of the core wrap or may be positioned intermediate the core wrap and the backsheet. The cushion material may have a basis weight in the range of about 20 gsm to about 40 gsm. The cushion material may comprise two or more layers of spunbond.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a plan view of an example absorbent article in the form of a taped diaper, garment-facing surface facing the viewer, in a flat laid-out state;

FIG. 2 is a plan view of the example absorbent article of FIG. 1, wearer-facing surface facing the viewer, in a flat laid-out state;

FIG. 3 is a front perspective view of the absorbent article of FIGS. 1 and 2 in a fastened position;

FIG. 4 is a front perspective view of an absorbent article in the form of a pant;

FIG. 5 is a rear perspective view of the absorbent article of FIG. 4;

FIG. 6 is a plan view of the absorbent article of FIG. 4, laid flat, with a garment-facing surface facing the viewer;

FIG. 7 is a cross-sectional view of the absorbent article taken about line 7-7 of FIG. 6;

FIG. 8 is a cross-sectional view of the absorbent article taken about line 8-8 of FIG. 6;

FIG. 9 is a plan view of an example absorbent core or an absorbent article;

FIG. 10 is a cross-sectional view, taken about line 10-10, of the absorbent core of FIG. 9;

FIG. 11 is a cross-sectional view, taken about line 11-11, of the absorbent core of FIG. 10;

FIG. 12 is an example of a bond pattern used on the outer cover materials of the present disclosure;

FIGS. 13-15 are example cross-sectional illustrations of various absorbent articles having outer cover materials and cushion materials of the present disclosure; and

FIG. 16 is a schematic view of a package of the present disclosure.

### DETAILED DESCRIPTION

Various non-limiting forms of the present disclosure will now be described to provide an overall understanding of the principles of the structure, function, manufacture, and use of the absorbent articles with cushiony softness disclosed herein. One or more examples of these non-limiting forms are illustrated in the accompanying drawings. Those of ordinary skill in the art will understand that the absorbent articles with cushiony softness described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

### General Description of an Absorbent Article

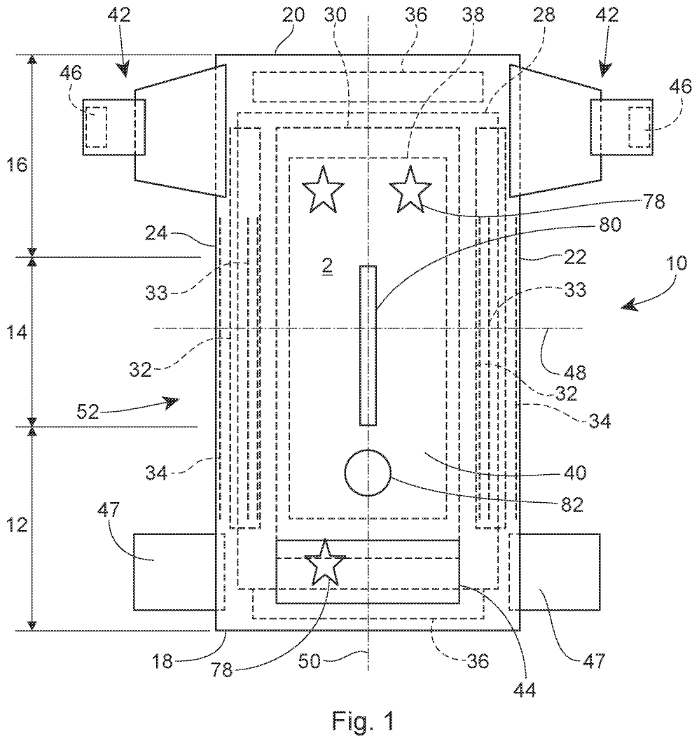

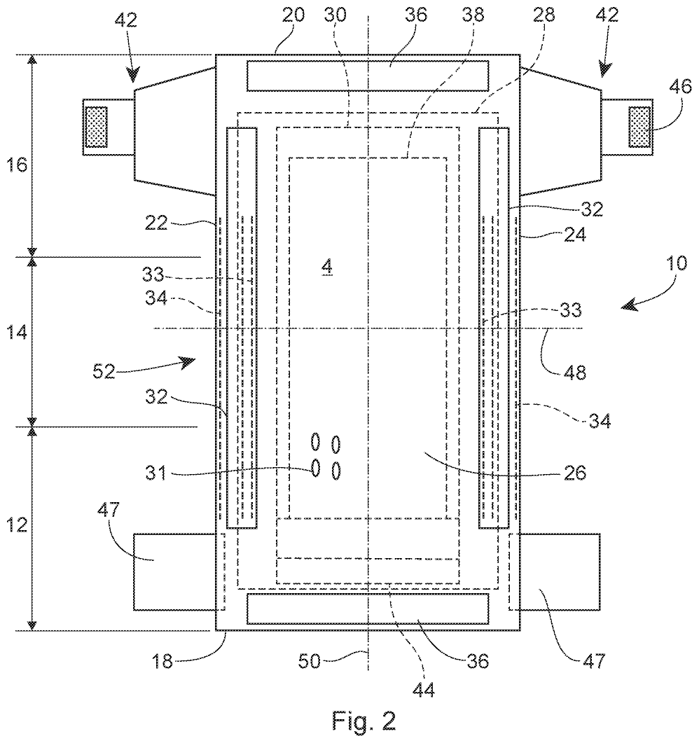

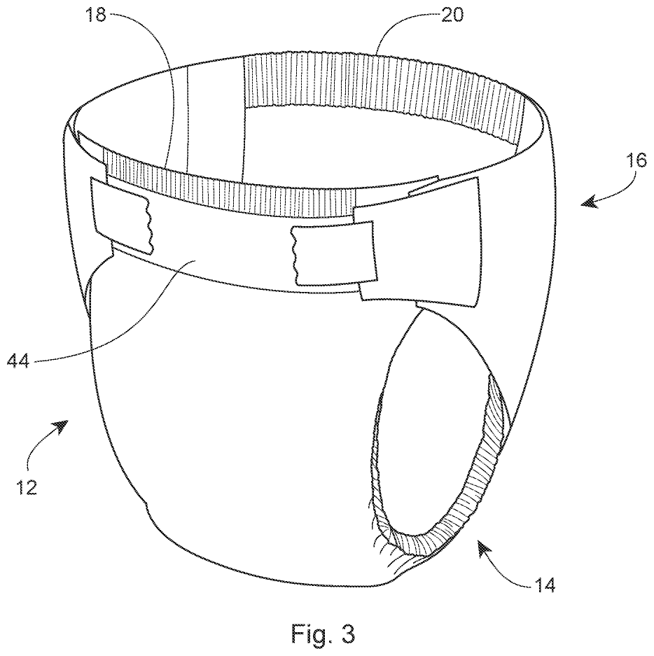

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as absorbent pads, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be ⅓ of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. An outer cover material 40, such as a nonwoven material, may cover a garment-facing side of the backsheet 28. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 in the front waist region 12. The absorbent article 10 may have a central lateral (or transverse) axis 48 and a central longitudinal axis 50. The central lateral axis 48 extends perpendicular to the central longitudinal axis 50.

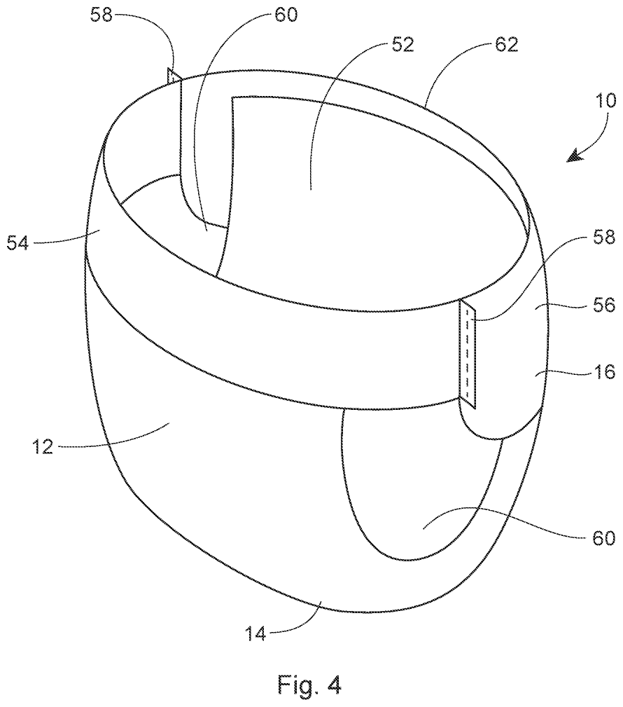

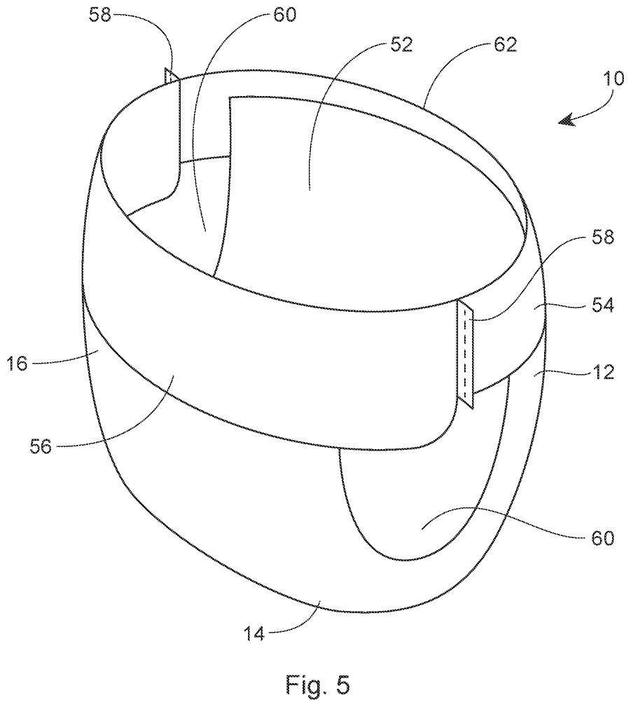

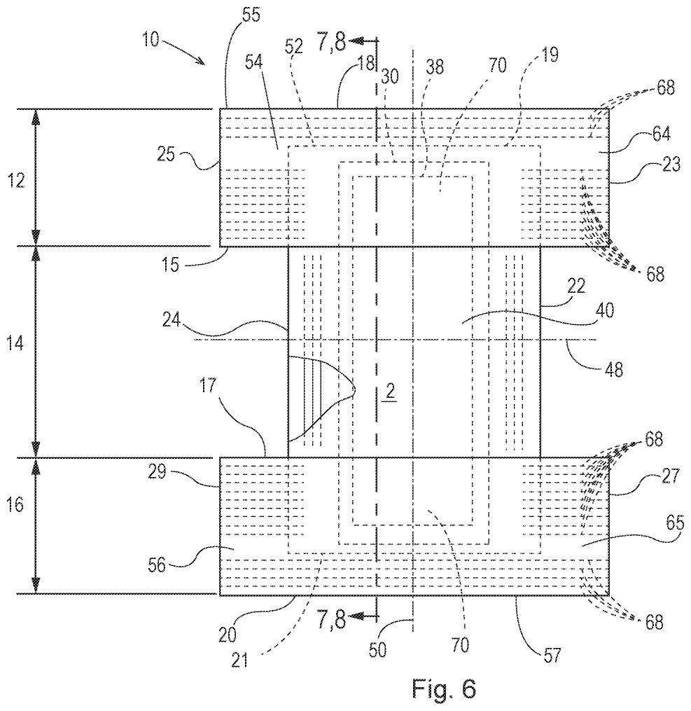

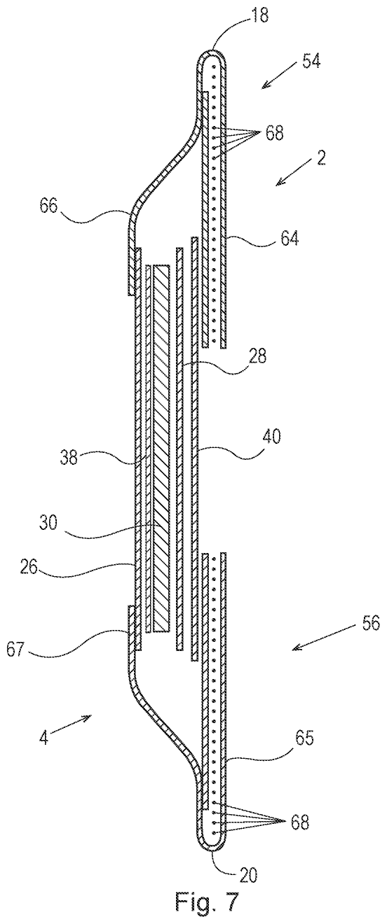

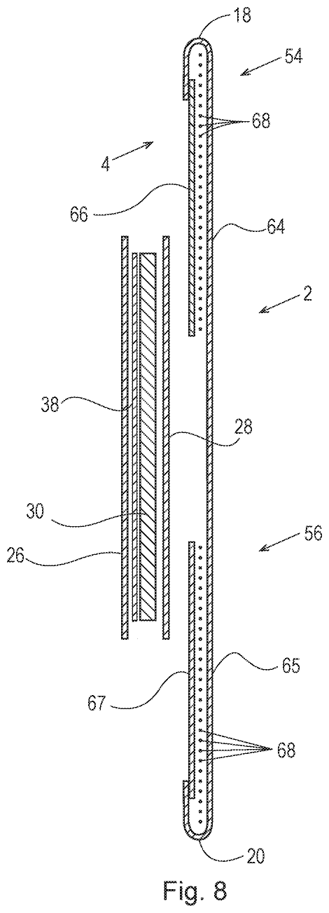

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137. Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56. The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. Appl. Pub. No. 2013/0211363.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 7 and a gap between the front and back inner belt layers 66 and 67 is shown in FIG. 8.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt seams 15 and 17; or, alternatively, adjacent to the seams 58, 15, and 17 in the manner described in U.S. Pat. No. 9,498,389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured (FIG. 2, element 31), may have any suitable three-dimensional features, and/or may have a plurality of embossments (e.g., a bond pattern). The topsheet may be apertured by overbonding a material and then rupturing the overbonds through ring rolling, such as disclosed in U.S. Pat. No. 5,628,097, to Benson et al., issued on May 13, 1997 and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials which permit vapors to escape from the absorbent article, while still preventing, or at least inhibiting, bodily exudates from passing through the backsheet.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features. The outer cover material 40 may be a hydroentangled nonwoven material with a bond pattern. The outer cover material 40 will be discussed in further detail below.

### Absorbent Core

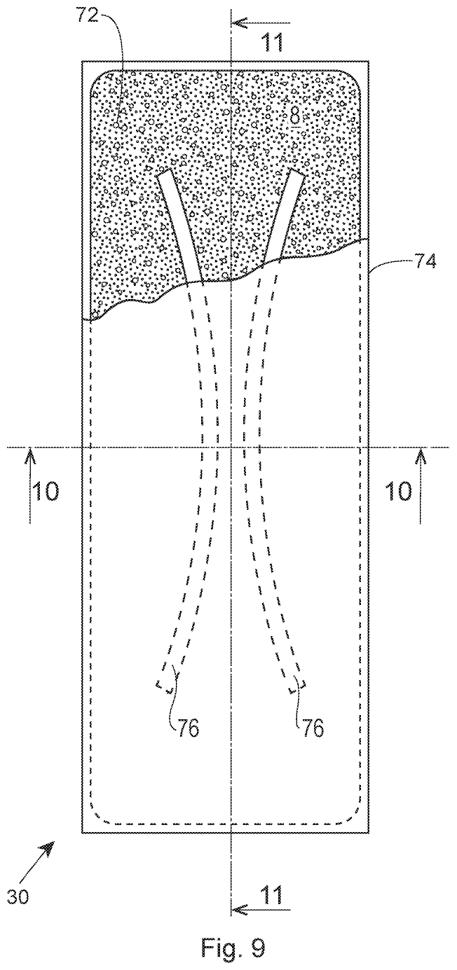

As used herein, the term “absorbent core” 30 refers to a component of the absorbent article 10 disposed in the article for absorbing and containing liquid such as urine received by the absorbent article. The absorbent core thus typically has a high absorbent capacity. An example absorbent core 30 is schematically shown in FIGS. 9-11. The absorbent core comprises an absorbent material 72, that is typically enclosed within or sandwiched between a core wrap 74.

The core wrap may be a single material that is folded and attached to itself, or it may comprise a separate top layer and bottom layer that may be bonded or otherwise joined together. The absorbent material typically comprises superabsorbent particles which are optionally mixed with cellulose fibers. As used herein, “absorbent core” does not include any acquisition-distribution systems, topsheet, or backsheet of the absorbent article.

The example absorbent core 30 shown in isolation in FIGS. 9-11 is in the dry state (before use). The absorbent core may typically have a generally rectangular shape as defined by its longitudinal edges and transversal front edge and back edge or may have other shapes.

Absorbent material 72 may be deposited as an absorbent layer having a generally rectangular outline, as represented in FIG. 9. A wide variety of absorbent cores may also be used. The absorbent material 72 layer may also have a non-rectangular perimeter (“shaped” core), in particular, the absorbent material 72 may define a tapering along its width towards the central region of the core (or “dog-bone” shape). In this way, the absorbent material deposition area may have a relatively narrow width in an area of the core intended to be placed in the crotch region of the absorbent article. This may provide for example better wearing comfort. Other shapes can also be used such as a “T” or “Y” or “hourglass” for the area of the absorbent material.

The absorbent material 72 may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. The absorbent material may also be free of cellulose fibers, as is known in so-called airfelt-free cores, where the absorbent material consists, or consists essentially, of SAP. The absorbent material may also be a high internal phase emulsion foam

“Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from 20 g/g to 40 g/g. “Superabsorbent polymer particles”, as used herein, refers to a superabsorbent polymer material which is in particulate form so as to be flowable in the dry state.

Various absorbent core designs comprising high amounts of SAP have been proposed in the past, see for example in U.S. Pat. No. 5,599,335 (Goldman), EP1,447,066 (Busam), WO95/11652 (Tanzer), U.S. Pat. Appl. Pub. No. 2008/0312622A1 (Hundorf), WO2012/052172 (Van Malderen). In particular, the SAP printing technology as disclosed in U.S. Pat. Appl. Pub. No. 2006/024433 (Blessing), U.S. Pat. Appl. Pub. No. 2008/0312617 and U.S. Pat. Appl. Pub. No. 2010/0051166A1 (both to Hundorf et al.) may be used. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate such as a carded nonwoven layer, having a porous structure into which SAP particles have been deposited, may also be used in present disclosure.

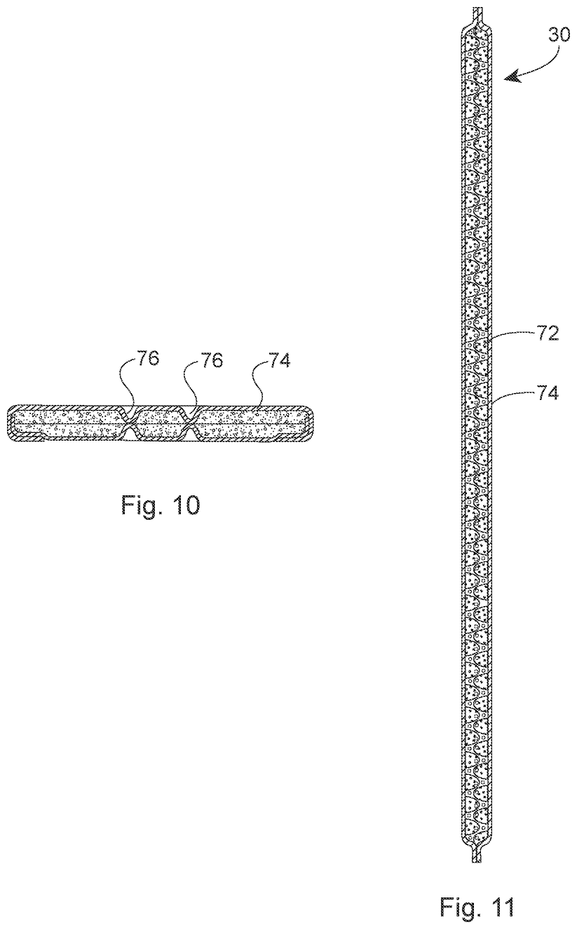

The absorbent material may be deposited as a continuous layer within the core wrap. The absorbent material may also be present discontinuously, for example, as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern, wherein the resulting layer is substantially continuously distributed across the absorbent particulate polymer material area, as illustrated in FIGS. 10-11. As for example taught in U.S. Pat. Appl. Pub. No. 2008/312,622A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core. The absorbent core may also comprise one or more longitudinally (or otherwise) extending channels 76, which are areas of the absorbent layer substantially free of absorbent material within the absorbent material layer. The top side of the core wrap may be advantageously bonded to the bottom side of the core by adhesive, mechanical or ultra-sonic bonding through these material-free areas. Example disclosures of such channels in an airfelt-free core can be found in WO2012/170778 (Rosati et al.) and US2012/0312491 (Jackels). Channels may of course also be formed in absorbent cores comprising a mix of cellulose fibers and SAP particles. These channels may embody any suitable shapes and any suitable number of channels may be provided. In other instances, the absorbent core may be embossed to create the impression of channels. The absorbent core in FIGS. 9-11 is merely an example absorbent core. Many other absorbent cores with or without channels are also within the scope of the present disclosure.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14.

### Elastic Waistband

Referring to FIGS. 1 and 2, the absorbent article 10 may comprise one or more elastic waistbands 36. The elastic waistbands 36 may be positioned on the garment-facing surface 2 or the wearer-facing surface 4. As an example, a first elastic waistband 36 may be present in the front waist region 12 near the front belt end edge 18 and a second elastic waistband 36 may be present in the back waist region 16 near the back end edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article.

### Acquisition Materials

Referring to FIGS. 1, 2, 7, and 8, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, cellulosic materials, cross-linked cellulosic materials, air laid cellulosic nonwoven materials, spunlace materials, or combinations thereof, for example. In some instances, portions of the acquisition materials 38 may extend through portions of the topsheet 26, portions of the topsheet 26 may extend through portions of the acquisition materials 38, and/or the topsheet 26 may be nested with the acquisition materials 38. Typically, an acquisition material 38 may have a width and length that are smaller than the width and length of the topsheet 26. The acquisition material may be a secondary topsheet in the feminine pad context. The acquisition material may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

### Landing Zone

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

### Wetness Indicator/Graphics

Referring to FIG. 1, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78.

### Front and Back Ears

Referring to FIGS. 1 and 2, as referenced above, the absorbent article 10 may have front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. The ears, or portions thereof, may be elastic or may have elastic panels. In an example, an elastic film or elastic strands may be positioned intermediate a first nonwoven material and a second nonwoven material. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2.

### Sensors

Referring again to FIG. 1, the absorbent articles of the present disclosure may comprise a sensor system 82 for monitoring changes within the absorbent article 10. The sensor system 82 may be discrete from or integral with the absorbent article 10. The absorbent article 10 may comprise sensors that can sense various aspects of the absorbent article 10 associated with insults of bodily exudates such as urine and/or BM (e.g., the sensor system 82 may sense variations in temperature, humidity, presence of ammonia or urea, various vapor components of the exudates (urine and feces), changes in moisture vapor transmission through the absorbent articles garment-facing layer, changes in translucence of the garment-facing layer, and/or color changes through the garment-facing layer). Additionally, the sensor system 82 may sense components of urine, such as ammonia or urea and/or byproducts resulting from reactions of these components with the absorbent article 10. The sensor system 82 may sense byproducts that are produced when urine mixes with other components of the absorbent article 10 (e.g., adhesives, agm). The components or byproducts being sensed may be present as vapors that may pass through the garment-facing layer. It may also be desirable to place reactants in the absorbent article that change state (e.g. color, temperature) or create a measurable byproduct when mixed with urine or BM. The sensor system 82 may also sense changes in pH, pressure, odor, the presence of gas, blood, a chemical marker or a biological marker or combinations thereof. The sensor system 82 may have a component on or proximate to the absorbent article that transmits a signal to a receiver more distal from the absorbent article, such as an iPhone, for example. The receiver may output a result to communicate to the caregiver a condition of the absorbent article 10. In other instances, a receiver may not be provided, but instead the condition of the absorbent article 10 may be visually or audibly apparent from the sensor on the absorbent article.

### Packages

The absorbent articles of the present disclosure may be placed into packages. The packages may comprise polymeric films and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages.

### Bio-Based Content for Components

Components of the absorbent articles described herein may at least partially be comprised of bio-based content as described in U.S. Pat. Appl. No. 2007/0219521A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

### Outer Cover Materials

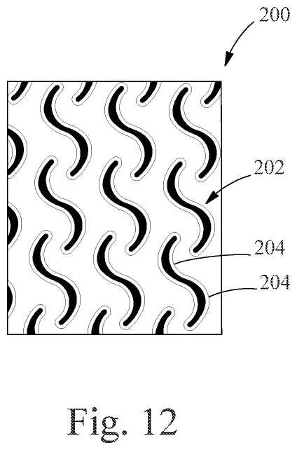

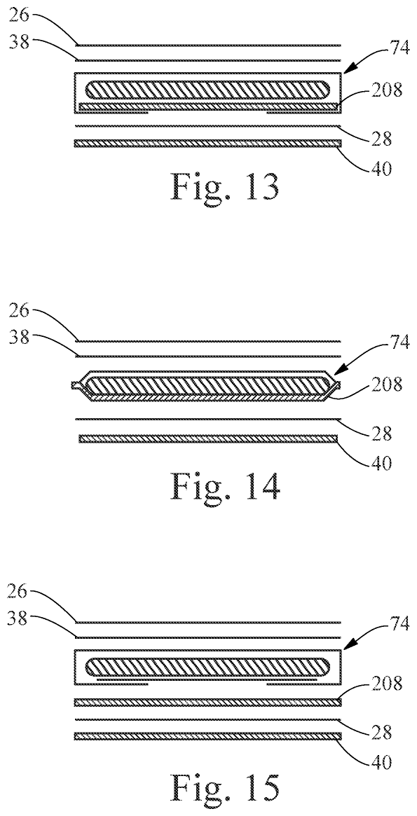

The outer cover materials of the present disclosure will now be described in further detail. The outer cover materials may comprise nonwoven hydroentangled spunbond materials and may comprise one or more spunbond layers, such as two (SS), three (SSS), or four (SSSS), for example. The hydroentangled spunbond materials are soft, robust, abrasion resistant, and strong. The outer cover materials may also not be hydroentangled, but still may comprise spunbond having one or more layers. The outer cover materials may comprise a softening agent, such as a slip additive like Vista Max, and other additives. Referring to FIG. 12, the outer cover materials may comprise a bond pattern 200 formed of bond elements 202. The bond pattern may have a bonded area of about 8% to about 17%, about 10% to about 16%, about 11% to about 15%, about 12% to about 14%, or about 13.9%, relative to an entire area of a surface of the outer cover materials, such as the garment-facing surface 2. In some forms, at least some of the bond elements may each comprise two arcuate portions 204. Other bond patterns and bond element shapes, sizes, and/or spacing are also contemplated within the scope of the present disclosure. Such other bond patterns may have the same bonded area as described above or different bonded areas. The outer cover materials 40 of the present disclosure may have a basis weight in the range of about 20 gsm to about 40 gsm, about 20 gsm to about 35 gsm, about 25 gsm to about 30 gsm, about 25 gsm, or about 30 gsm, according to the Basis Weight Test herein. Example outer cover materials are illustrated in FIGS. 13-15. The high basis weight, hydroentangled outer cover materials with the bond patterns provide a soft and cushiony feel to the absorbent articles. The outer cover materials may have a thickness in the range of about 0.15 mm to about 0.5 mm, about 0.18 mm to about 0.25 mm, or about 0.30 mm to about 0.34 mm.

### Kinetic Sliding Force of Outer Cover Material

A garment-facing surface 2 of the outer cover materials 40 of the present disclosure may have a Kinetic Sliding Force in the range of about 6.0 to about 7.5, about 6.5 to about 7.5, about 6.5 to about 7.0, or about 6.7, according to the Kinetic Sliding Force Test herein. The outer cover materials tested were 30 gsm with the bond pattern illustrated in FIG. 12. A comparative example (current market product, Pampers Swaddlers) was 19 gsm and had the bond pattern of FIG. 12. The comparative example had a Kinetic Sliding Force of 4.5, according to the Kinetic Sliding Force Test herein. Therefore, the outer cover materials of the present disclosure have a significantly higher Kinetic Sliding Force, but still provide the benefit of cushiony softness and loft.

As mentioned above, absorbent articles are at times packaged under significant compression. Therefore, it may be difficult to dispense the first few absorbent articles from packages. The outer cover materials of the present disclosure through their composition, texture, and bond patterns may enhance absorbent article dispensing, especially from a full and compressed package of absorbent articles. This is true for the absorbent articles of the present disclosure even though the absorbent articles of the present disclosure have outer cover materials having a higher Kinetic Sliding Force (i.e., about 6 to about 7.5) as current outer cover materials. As such, Applicants have broken a technical barrier by raising the Kinetic Sliding Force of the outer cover materials while still achieving better dispensing from a compressed package of absorbent articles and maintaining a pronounced texture.

### In-Bag Stack Height

To aid in dispensing from a package and maintain loft and cushiony softness, the compression of the absorbent articles, characterized as stack height of the packages may be increased. The present inventors have increased the stack height to achieve the benefits discussed above. The increase in stack height reduced compression forces applied to the absorbent articles by the package by about 15% to about 25%, about 20%, about 21%, about 22%, about 23%, or about 25%, specifically reciting all 0.1% increments within the specified ranges and all ranges formed therein or thereby. Packages of the absorbent articles of the present disclosure may have an In-Bag Stack Height of less than about 115 mm, less than about 110 mm, less than about 105 mm, or less than about 103 mm, but greater than about 70 mm, greater than about 83 mm, greater than about 90 mm, greater than about 95 mm, or greater than about 100 mm, specifically reciting all 0.1 mm increments within the specified ranges and all ranges formed therein or thereby, according to the In-Bag Stack Height Test described herein. Packages of the absorbent articles of the present disclosure may have an In-Bag Stack Height in the range of about 70 mm to about 110 mm, about 80 mm to about 110 mm, about 83 mm to about 110 mm, about 90 mm to about 110 mm, about 95 mm to about 105 mm, about 93 mm to about 105 mm, about 95 mm to about 103 mm, or about 96 mm to about 102 mm, specifically reciting all 0.1 mm increments within the specified ranges and all ranges formed therein or thereby, according to the In-Bag Stack Height Test described herein.

### Cushion Material

One or more cushion materials may be provided in the absorbent articles 10 intermediate the topsheet and the backsheet. A cushion material may be a layer, laminate, or material that provides a cushiony feel when the absorbent article is touched from the garment-facing surface 2 or the wearer-facing surface 4. The cushion material may “mask” a grainy feel potentially caused by the absorbent material 72, such as superabsorbent polymers. The cushion material may also “mask” bodily exudates from being visible when viewing the wearer-facing surface 4 or the garment-facing surface 2 of the absorbent article 10. The cushion material may comprise nonwoven hydroentangled spunbond materials and may comprise one or more spunbond layers, such as two (SS), three (SSS), or four (SSSS), for example. The cushion material may also not be hydroentangled, but still may comprise one more spunbond layers. The cushion material may have a basis weight in the range of about 15 gsm to about 50 gsm, about 20 gsm to about 45 gsm, about 25 gsm to about 40 gsm, about 30 to about 40 gsm, about 25 gsm, about 30 gsm, about 35 gsm, or about 40 gsm, according to the Basis Weight Test herein. The cushion material may be the layer forming the garment-facing side or the wearer-facing side of the core bag 74. The cushion material may also be placed within the core bag 74. The cushion material may be a separate material positioned intermediate the garment-facing side of the core bag 74 and the liquid impermeable backsheet 28. Referring to FIG. 13, a cushion material 208 may form all of, or at least a portion of, the garment-facing layer of the core bag 74, with the core bag 74 having a C-wrap configuration. Referring to FIG. 14, a cushion material 208 may form all of, or at least a portion of, the garment-facing layer of the core bag 74, with the core bag not having a C-wrap configuration. Referring to FIG. 15, a cushion material 208 may be positioned intermediate a garment-facing surface of the core bag 74 and the backsheet 28. The cushion material may be free of a softening additive or may comprise a softening additive. The cushion materials, in combination with the outer cover materials discussed herein provides the absorbent articles with cushiony softness and loft. The cushion materials may have a thickness in the range of about 0.15 mm to about 0.5 mm, about 0.25 mm to about 0.30 mm, or about 0.35 mm to about 0.40 mm, specifically reciting all 0.1 mm increments within the specified ranges and all ranges formed therein or thereby. The hydroentangled cushion materials are quite flexible compared to non-hydroentangled spunbond at the same basis weight. The cushion materials may have indicia printed, or otherwise applied, thereon.

### Energy of Compression

Energy of Compression of the cushion material is illustrated in Table 1 below. All data was generated according to the Energy of Compression Test herein.

TABLE 1
Energy at Percent Compression

| | Energy of | | Energy of | |
|---|---|---|---|---|
| | Compression: | | Compression: | |
| | Pampers Swaddlers | | Present | |
| | Current market | | Disclosure | |
| | product, garment- | | garment-facing | |
| | facing nonwoven of | | nonwoven of | |
| | core wrap, 10 gsm | Base | core wrap, 35 | Base |
| Sample | (Comparative Example) | size | gsm | size |
| 20% | 2.1 | 9 | 9.2 | 11 |
| Compression | | | | |
| 25% | 3.0 | 9 | 13.6 | 11 |
| Compression | | | | |
| 30% | 4.5 | 9 | 20.5 | 11 |
| Compression | | | | |
| 50% | 24.8 | 9 | 113.4 | 9 |
| Compression | | | | |

Energy units—μJ
Compression units—% of thickness

As can be seen from the above, the energy (μJ) required to compress the inventive cushion material at 20%, 25%, 30%, and 50% compression is much greater than the energy (μJ) required to compress the comparative example material at the same levels of compression. This indicates that the cushion material provides loft and thickness in an absorbent article, when used a garment-facing nonwoven of a core wrap or when positioned intermediate the garment-facing nonwoven of a core wrap and a backsheet film. The cushion material may be used to mask grainy portions of superabsorbent material in an absorbent core or to mask other materials.

### Emtec

The present disclosure provides outer cover materials with cushiony softness even with a high Kinetic Sliding Force. Softness, texture (i.e., smoothness), and/or stiffness may be measured by an Emtec Tissue Softness Analyzer, according to the Emtec Test herein. Tactile softness is measured as TS7. Texture/Smoothness is measured as TS750. Stiffness is measured as D.

A portion of, or all of, a garment-facing surface of the outer cover materials 40 of the present disclosure may have a TS7 value in the range of about 1 dB V² rms to about 4.5 dB V² rms, about 2 dB V² rms to about 4.5 dB V² rms, or about 2 dB V² rms to about 4.0 dB V² rms. The portion of, or all of, the garment-facing surface of the outer cover materials 40 may also have a TS750 value in the range of about 4 dB V² rms to about 30 dB V² rms, about 6 dB V² rms to about 30 dB V² rms, about 6 dB V² rms to about 20 dB V² rms, about 6 dB V² rms to about 15 dB V² rms, about 6 dB V² rms to about 12 dB V² rms, or about 6.5 dB V² rms to about 10 dB V² rms. The portion of, or all of, the garment-facing surface of the outer cover materials 40 may also have a D value in the range of about 1 mm/N to about 10 mm/N, about 3 mm/N to about 8 mm/N, about 2 mm/N to about 6 mm/N, about 2 mm/N to about 4 mm/N, or about 3 mm/N to about 4 mm/N. All values are measured according to the Emtec Test herein. The TS7 value is tactile softness, so low numbers are desired (the lower the number, the more soft the material is). The TS750 value is texture so a high number is desired (the higher the number, the more texture the material has). Having a low TS7 value and a high texture value is contradictory in that typically the more texture a nonwoven fabric has, the less soft it is. The wearer-facing surface of the outer cover materials 40 may have a different TS7, TS750, or D value as the garment-facing surface.

It may be desirable to have the certain TS7 and TS750 properties discussed above in both the outer cover nonwoven material and a topsheet. This provides soft texture on both sides (i.e., wearer-facing and garment-facing) of the absorbent article.

### Test Methods

### In-Bag Stack Height Test

The In-Bag Stack Height of a package of absorbent articles is determined as follows:

### Equipment

A thickness tester with a flat, rigid horizontal sliding plate is used. The thickness tester is configured so that the horizontal sliding plate moves freely in a vertical direction with the horizontal sliding plate always maintained in a horizontal orientation directly above a flat, rigid horizontal base plate. The thickness tester includes a suitable device for measuring the gap between the horizontal sliding plate and the horizontal base plate to within ±0.5 mm. The horizontal sliding plate and the horizontal base plate are larger than the surface of the absorbent article package that contacts each plate, i.e., each plate extends past the contact surface of the absorbent article package in all directions. The horizontal sliding plate exerts a downward force of 850±1 gram-force (8.34 N) on the absorbent article package, which may be achieved by placing a suitable weight on the center of the non-package-contacting top surface of the horizontal sliding plate so that the total mass of the sliding plate plus added weight is 850±1 grams.

### Test Procedure

Absorbent article packages are equilibrated at 23±2° C. and 50±5% relative humidity prior to measurement.

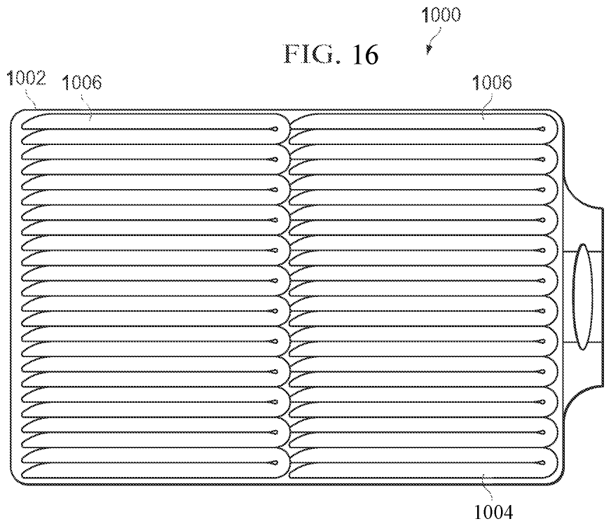

The horizontal sliding plate is raised and an absorbent article package is placed centrally under the horizontal sliding plate in such a way that the absorbent articles within the package are in a horizontal orientation (see FIG. 16). Any handle or other packaging feature on the surfaces of the package that would contact either of the plates is folded flat against the surface of the package so as to minimize their impact on the measurement. The horizontal sliding plate is lowered slowly until it contacts the top surface of the package and then released. The gap between the horizontal plates is measured to within ±0.5 mm ten seconds after releasing the horizontal sliding plate. Five identical packages (same size packages and same absorbent articles counts) are measured and the arithmetic mean is reported as the package width. The “In-Bag Stack Height”=(package width/absorbent article count per stack)×10 is calculated and reported to within ±0.5 mm.

### Emtec Test

The Emtec Test is performed on portions of interest of outer cover nonwoven materials or topsheets. In this test, TS7, TS750, and D values are measured using an Emtec Tissue Softness Analyzer (“Emtec TSA”) (Emtec Electronic GmbH, Leipzig, Germany) interfaced with a computer running Emtec TSA software (version 3.19 or equivalent). The Emtec TSA includes a rotor with vertical blades which rotate on the test sample at a defined and calibrated rotational speed (set by manufacturer) and contact force of 100 mN. Contact between the vertical blades and the test sample creates vibrations both in the blades and in the test piece, and the resulting sound is recorded by a microphone within the instrument. The recorded sound file is then analyzed by the Emtec TSA software to determine TS7 and TS750 values. The D value is a measure of sample stiffness and is based on the vertical distance required for the contact force of the blades on test sample to be increased from 100 mN to 600 mN. The sample preparation, instrument operation, and testing procedures are performed according the instrument manufacturer's specifications.

### Sample Preparation

A test sample is prepared by cutting a square or circular portion of interest from the outer cover nonwoven material or topsheet of an absorbent article. It is preferable that freeze spray is not used to remove the portion of the outer cover nonwoven material or topsheet to be analyzed, though it is acceptable to use freeze spray in a distal region to aid in initiating the separation of layers. Test samples are cut to a length and width (diameter in the case of a circular sample) of no less than about 90 mm and no greater than about 120 mm to ensure the sample can be clamped into the TSA instrument properly. (If an absorbent article does not contain a sufficiently large area of the substrate of interest to extract a sample of the size specified above, it is acceptable to sample equivalent material from roll stock.) Test samples are selected to avoid unusually large creases or folds within the testing region. Six substantially similar replicate samples are prepared for testing.

All samples are equilibrated at TAPPI standard temperature and relative humidity conditions (23° C.±2 C.° and 50%±2%) for at least 2 hours prior to conducting the TSA testing, which is also conducted under TAPPI conditions.

### Testing Procedure

The instrument is calibrated according to the Emtec's instructions using the 1-point calibration method with the appropriate reference standards (so-called “ref.2 samples,” or equivalent, available from Emtec).

A test sample is mounted in the instrument with the surface of interest facing upward, and the test is performed according to the manufacturer's instructions. The software displays values for TS7, TS750, and D when the automated instrument testing routine is complete. TS7 and TS750 are each recorded to the nearest 0.01 dB V² rms, and D is recorded to the nearest 0.01 mm/N. The test sample is then removed from the instrument and discarded. This testing procedure is performed individually on the corresponding surfaces of interest of each of the six of the replicate samples (wearer-facing surface for topsheet samples and garment-facing surface for outer cover nonwoven material samples).

The value of TS7, TS750, and D are each averaged (arithmetic mean) across the six sample replicates. The average values of TS7 and TS750 are reported to the nearest 0.01 dB V² rms. The average value of D is reported to the nearest 0.01 mm/N.

### Basis Weight Test

Basis weight of the outer cover materials and the cushion materials described herein may be determined by several available techniques, but a simple representative technique involves taking an absorbent article or other consumer product, removing any elastic which may be present and stretching the absorbent article or other consumer product to its full length. A punch die having an area of 45.6 cm² is then used to cut a piece of the outer cover material or cushion layer from the approximate center of the absorbent article or other consumer product in a location which avoids to the greatest extent possible any adhesive which may be used to fasten the outer cover materials or the cushion materials to any other layers which may be present and removing the outer cover materials or cushion material from other layers (using cryogenic spray, such as Cyto-Freeze, Control Company, Houston, Tex., if needed). The sample is then weighed and dividing by the area of the punch die yields the basis weight of the patterned apertured web. Results are reported as a mean of 5 samples to the nearest 0.1 cm².

### Kinetic Sliding Force Test

The kinetic sliding force of a nonwoven material is determined using the measurement approach described in ASTM D1894-14 with the following additional specifics and modifications. The sled apparatus used has a stationary plane and a moving sled and is that depicted in FIG. 1(c). A constant-rate-of-extension tensile tester is typically used to facilitate the movement and force measurement required. The sled is 4.0 inches×4.0 inches and is moved at a speed of 900±30 mm/min and the travel length is 100±2 mm. The total weight of the wrapped sled and attached specimen is 1000±5 g. The stationary plane used is a polished stainless-steel plate having a nondirectional, mirror-like finish of appearance number 8.

Five nominally equivalent specimens of sample material are analyzed. If the sample material is available as raw-material roll stock, each specimen is a rectangle 5.0 inches in the roll's machine direction (MD) and 4.5 inches in the roll's cross direction (CD). The surface to be analyzed is the outer surface of the roll. If the sample material is harvested from finished absorbent articles, the nonwoven of interest is first separated from five like absorbent articles, optionally using cryogenic freeze spray, so as to minimally perturb the nonwoven. The dimension of the nonwoven that was parallel to the central longitudinal axis of the absorbent article is defined as the specimen MD, and the specimen is trimmed to 5.0 inches in this dimension. The dimension of the nonwoven that was parallel to the central lateral axis of the article is defined as the specimen CD, and the specimen is trimmed to 4.5 inches in this dimension. When trimming specimens in this way, each is cut so as to be centered at the point that was located at the intersection of the central lateral and central longitudinal axes of the absorbent article.

To analyze a specimen, it is first affixed to the sled such that the MD axis of the specimen is parallel to the direction of sled travel. To facilitate secure attachment of nonwoven substrate to the sled, hook material is attached to the upper surface of the sled at the sides and leading edge. The sled is moved at a speed of 900±30 mm/min and travel length is 100±2 mm, and force data are collected in Newtons (N) with a precision of at least 0.1 N and at 100 Hz. (A load cell is chosen such that the measured force is between 10% and 90% of the load cell's range.) For each specimen, the average (arithmetic mean) measured force data from 10 mm to 95 mm of crosshead displacement is calculated, and this is recorded as the kinetic sliding force of that specimen. The arithmetic mean of the five values of kinetic sliding force corresponding to each of the five analyzed specimens is calculated and reported to the nearest 0.1 N as the Kinetic Sliding Force for the nonwoven material.

### Energy of Compression Test

The Energy of Compression Test makes use of a controlled-stress compression instrument to determine the energy required to compress a sample material in thickness to one or more relative compression extents of interest. Force vs. displacement data are converted into energy vs. relative compression data, and a linear interpolation is performed to determine the energy corresponding to relative compression. Specimens are equilibrated for at least 24 hours to and measured at 25±2° C.

A suitable instrument is a controlled-stress apparatus that is: (1) capable of operating in compression mode, (2) capable of controlling and measuring force with a precision of 0.001 N (or better) up to at least 0.87 N, (3) capable of measuring displacement with a precision of 0.001 mm (or better), and (4) capable of sampling both force and position at least at 600 Hz. The tooling used in this method are two parallel metal plates 15 mm in diameter. One suitable example of a such an instrument is the Q800 Dynamic Mechanical Analyzer (DMA) available from TA Instruments, New Castle, Del., USA.

Sample material of interest is preferably analyzed in raw-material, roll-stock form. Specimens are circles of 12.7 mm diameter. If raw material is not available, sample material specimens may be excised from one or more like finished-product absorbent articles. Excision of specimens must be performed in a way so as to minimally perturb the properties of the sample being probed by this method. Excision will generally include cutting and may include the use of cryogenic freeze spray to separate sheets joined by adhesive. It may also be necessary to wash entire absorbent articles or nascent specimens in an appropriate solvent, such as tetrahydrofuran, to dissolve and remove all adhesive residue. Regardless of whether specimens are taken from raw material or finished product, 10±1 specimens are excised and analyzed.

For each specimen analyzed, the parallel plates of the controlled-stress compression instrument are separated, and the specimen is placed centered on the lower plate. (The position of the plates are referenced to the state in which they are touching, which is defined as “zero” corresponding to “zero thickness.”) The instrument is then ramped up in force load at a rate of 2.000 N/min up to a load of 0.005 N where it is held constant for 6 seconds. This 6-second hold is referred to as the specimen equilibration step, and the position of the plates at the end of the 6-second time span is recorded to be the starting thickness, to, of the specimen, to the nearest 0.001 mm. Immediately after the 6-s equilibration step, the instrument is used to continue increasing the load at 2.000 N/min to 0.87 N (1.0 psi). This latter step is referred to as the compression step. In the compression step, force is recorded with precision 0.001 N or better and position is recorded with precision 0.001 mm or better, and both are recorded at 600 Hz.

For each specimen, the nascent data file from the compression step consists of force vs. position data. Each data point in this data set is converted to energy of compression vs. relative compression data. The relative compression is defined as

$$relative compression = \frac{{t}_{0} - {t}_{n}}{{t}_{0}} \times 100 \% ,$$

where tₙ is the position (thickness) measured nth data point. The energy of compression of the nth data point is the integral of force vs. position from the first data point in the compression step up to and including the nth data point. For each specimen, the energy corresponding to a relative compression level of interest (e.g., 20%, 25%, 30%, 50%, etc.) is generally determined by linear interpolation. That is, if no relative compression data point exists exactly corresponding to the relative compression of interest, the two data points immediately proximal to the relative compression of interest (above and below) are used to determine the energy of compression corresponding to the relative compression of interest through simple linear interpolation. If all points in the compression step data exhibit a relative compression less than the relative compression of interest, the energy of compression corresponding to the highest value of relative compression in the compression step is used.

For a sample material and for any relative compression of interest, the arithmetic mean of the energy of compressions for all specimens analyzed for that sample material is calculated and reported in units of μJ to the nearest 0.1 μJ as the Energy of Compression for the sample material at the relative compression of interest.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this present disclosure.

What is claimed is:

1. An absorbent article comprising:
a liquid permeable topsheet;
a liquid impermeable backsheet;
an absorbent core positioned at least partially intermediate the topsheet and the backsheet;
the absorbent core positioned within a core wrap, the absorbent core comprising an absorbent material; and
a hydroentangled outer cover material joined to the backsheet and forming a portion of a garment-facing surface of the absorbent article;
wherein the outer cover material comprises two or more layers of spunbond;
wherein the outer cover material comprises a bond pattern formed of bond elements,
wherein the bond pattern has a bonded area of about 8% to about 17% relative to an entire area of a surface of the outer cover material;
wherein the spunbond of the outer cover material comprises a softening additive; and
wherein the outer cover material has a basis weight in the range of about 20 gsm to about 35 gsm, according to the Basis Weight Test; and
a hydroentangled cushion material positioned intermediate the backsheet and the absorbent material;
wherein the cushion material has a basis weight in the range of about 20 gsm to about 40 gsm, according to the Basis Weight Test; and
wherein the cushion material comprises two or more layers of spunbond.

2. The absorbent article of claim 1, wherein the cushion material forms at least a portion of a garment-facing surface of the core wrap.

3. The absorbent article of claim 1, wherein the cushion material is positioned intermediate the backsheet and a garment-facing surface of the core wrap.

4. The absorbent article of claim 1, wherein the cushion material comprises four layers of the spunbond, and wherein the outer cover material comprises four layers of the spunbond.

5. The absorbent article of claim 1, wherein the cushion material is free of a softening additive.

6. The absorbent article of claim 1, wherein at least some of the bond elements each comprise two arcuate portions.

7. The absorbent article of claim 1, wherein the outer cover material has a basis weight of about 25 gsm to about 30 gsm, according to the Basis Weight Test.

8. The absorbent article of claim 1, wherein a garment-facing surface of the outer cover material has a Kinetic Sliding Force in the range of about 6.0 to about 7.5, according to the Kinetic Sliding Force Test.

9. An absorbent article comprising:
a liquid permeable topsheet;
a liquid impermeable backsheet;
an absorbent core positioned at least partially intermediate the topsheet and the backsheet;
the absorbent core positioned within a core wrap, the absorbent core comprising an absorbent material; and
a hydroentangled outer cover material joined to the backsheet and forming a portion of a garment-facing surface of the absorbent article;
wherein the outer cover material comprises three or more layers of spunbond;
wherein the outer cover material comprises a bond pattern formed of bond elements,
wherein the bond pattern has a bonded area of about 8% to about 17% relative to an entire area of a surface of the outer cover material;
wherein the spunbond of the outer cover material comprises a softening additive;
wherein the outer cover material has a basis weight in the range of about 20 gsm to about 35 gsm, according to the Basis Weight Test; and
wherein a garment-facing surface of the outer cover material has a Kinetic Sliding Force in the range of about 6.0 to about 7.5, according to the Kinetic Sliding Force Test.

10. The absorbent article of claim 9, comprising a hydroentangled cushion material positioned intermediate the backsheet and the absorbent material;
wherein the cushion material has a basis weight in the range of about 20 gsm to about 40 gsm, according to the Basis Weight Test; and
wherein the cushion material comprises three or more layers of spunbond.

11. The absorbent article of claim 10, wherein the cushion material forms at least a portion of a garment-facing surface of the core wrap.

12. The absorbent article of claim 10, wherein the cushion material is positioned intermediate the backsheet and a garment-facing surface of the core wrap.

13. The absorbent article of claim 10, wherein the cushion material comprises four layers of the spunbond, wherein the outer cover material comprises four layer of the spunbond, and wherein the cushion material is free of a softening additive.

14. The absorbent article of claim 9, wherein at least some of the bond elements each comprise two arcuate portions, and wherein the outer cover material has a basis weight of about 25 gsm of about 30 gsm, according to the Basis Weight Test.

15. A package comprising the absorbent article of claim 9, wherein the package has an In-Bag Stack Height in the range of about 95 mm to about 110 mm, according to the In-Bag Stack Height Test.

16. An absorbent article comprising:
a liquid permeable topsheet;
a liquid impermeable backsheet;
an absorbent core positioned at least partially intermediate the topsheet and the backsheet;
the absorbent core positioned within a core wrap, the absorbent core comprising an absorbent material; and
a hydroentangled outer cover material joined to the backsheet and forming a portion of a garment-facing surface of the absorbent article;
wherein the outer cover material comprises three or more layers of spunbond;
wherein the outer cover material comprises a bond pattern formed of bond elements, wherein at least some of the bonds comprise two arcuate portions.
wherein the bond pattern has a bonded area of about 10% to about 16% relative to an entire area of a surface of the outer cover material;
wherein the spunbond of the outer cover material comprises a softening additive;
wherein the outer cover material has a basis weight in the range of about 20 gsm to about 35 gsm, according to the Basis Weight Test; and
wherein a garment-facing surface of the outer cover material has a Kinetic Sliding Force in the range of about 6.0 to about 7.5, according to the Kinetic Sliding Force Test; and
a hydroentangled cushion material forming at least a portion of a garment-facing surface of the core wrap;
wherein the cushion material has a basis weight in the range of about 20 gsm to about 40 gsm, according to the Basis Weight Test; and
wherein the cushion material comprises three or more layers of spunbond.

17. The absorbent article of claim 16, wherein the absorbent material is substantially free of cellulosic fibers.

18. The absorbent article of claim 16, wherein the garment-facing surface of the outer cover material has a TS7 value in the range of about 1 dB V² rms to about 4.5 dB V² rms, according to the Emtec Test.

19. The absorbent article of claim 18, wherein the garment-facing surface of the outer cover material has a TS750 value in the range of about 6 dB V² rms to about 30 dB V² rms, according to the Emtec Test.

20. The absorbent article of claim 16, wherein cushion material has an energy of compression of about 9.2 μJ at 20% compression, according to the Energy of Compression Test.

21. A package comprising the absorbent article of claim 22, wherein the package has an In-Bag Stack Height in the range of about 95 mm to about 110 mm, according to the In-Bag Stack Height Test.
